(19) United States
(12) Patent Application Publication
(10) Pub. No.: US 2016/0287113 A1
(43) Pub. Date: Oct. 6, 2016

(54) MICROELECTRODES MADE FROM STRUCTURED DIAMOND FOR NEURAL INTERFACING APPLICATIONS

(71) Applicant: COMMISSARIAT A L'ENERGIE ATOMIQUE ET AUX ENERGIES ALTERNATIVES, Paris (FR)

(72) Inventors: Clément Hebert, Fonville (FR); Emmanuel Scorsone, Magny-les-Hameaux (FR); Jean-Paul Mazellier, Poissy (FR); Lionel Rousseau, Le Perreux-sur-Marne (FR)

(73) Assignee: COMMISSARIAT A L'ENERGIE ATOMIQUE ET AUX ENERGIES ALTERNATIVES, Paris (FR)

(21) Appl. No.: 15/029,607

(22) PCT Filed: Oct. 14, 2014
(86) PCT No.: PCT/IB2014/065305

§ 371 Date: Apr. 14, 2016

(30) Foreign Application Priority Data
Oct. 16, 2013 (FR) 1360074

(51) Int. Cl. A61B 5/04; A61N 1/05

(52) U.S. Cl. CPC A61B 5/04001; A61N 1/0551; A61B 2562/125

(57) ABSTRACT

A microelectrode (2) for neural interfacing applications comprises a first substrate layer (4), a second attachment layer (6), and a third layer (8) forming the active part of the electrode (2) of which the material consists of synthetic diamond made in electrically conductive by doping with atoms chosen from boron, nitrogen and phosphorus atoms. The material of the third layer (8) is a textured material that comprises a compact assembly, in the form of a brush, of tubes (26) each comprising, in the form of at in least one peripheral outer layer, polycrystalline diamond made electrically conductive by doping. The tubes (26) are separated from each other at the first fixed ends (28) of same and project the free ends (30) of same away from the first and second layers (4, 6) in a direction that is substantially vertical relative to the extension plane (20) of the second layer (6). A method for producing said microelectrode is also described.

The present invention relates to microelectrodes and to devices using these electrodes for neural interfacing applications as well as methods for manufacturing such electrodes and electrode devices. These electrodes may be used both for neurostimulation or for recording electric signals called “Action Potentials” (PA). In the first case, one or several electric pulses are transmitted from the electrode to neuronal cells in contact or in proximity to the electrode. The idea here is to often activate a defective neurophysiological function via an implantable medical device. Known examples of applications are notably cochlear implants, retinal implants, deep brain stimulation, cortical implants. In the second case, electric signals (AP), propagating through a neuronal network in contact or in proximity to the electrodes, are measured. The sought applications here in particular relate to electro-physiological studies. The devices, used in these applications, are known under the name of multi-electrode arrays (MEA), and give the possibility of studying ex-vivo in the laboratory the electric activity of neurones.

The electrodes used for neurostimulation have to meet well-defined requirements. First of all, the material making up the electrode has to be biocompatible. Further, the charge transfer process should not generate necrosis of tissues. Thus, the charge transfer has to be made in a capacitive way or by a reversible Faradic transfer involving redox species present at the surface of the electrode. This Faradic transfer of charges of course should not generate toxic species, gas bubbles or a significant pH variation. In spite of these constraints, the injected charge from the electrode to the tissues should attain a density of typically several mC.cm⁻² in order to be efficient. Finally, the electrode should be robust for implantation in vivo. In particular, it should include good mechanical strength and chemical inertia in order to withstand ageing upon contact with tissues and should retain its surface condition after implantation.

If the biocompatibility is less critical in the case of the MEA systems used ex-vivo in the field of electrophysiology, the electrodes should however have electrochemical properties close to those of the electrodes used for neurostimulation. In the case of their use for neuronal recording, they should moreover have a low electrochemical impedance, typically less than 300 kOhms in order to obtain sufficiently large signal-to-noise level ratios. This impedance is strongly related to the diameter of the electrode. The development of these systems urges the use of electrodes having increasingly small diameters in order to obtain better spatial resolution of the neuronal activities. Now, with constant electrode structure, the reduction of the diameter increases the impedance. In order to overcome this drawback, artificially increasing the surface area of the electrodes is known.

Moreover, when such MEA devices are used for neurostimulation, one skilled in the art is confronted with the problem of gradual degradation of the electrode related to the passing of current through the electrode. This is particularly critical in the case of systems including electrodes as thin layers (with a thickness typically less than 5 micrometers) since, over time, there exists a significant risk of complete disappearance of the electrode material. Thus the electro-physiologist is sometimes upon using metal electrodes led to proceeding with electro-deposition of metal in order to regenerate the electrodes after a few uses in order to be able to again find their initial performances.

A condition for obtaining both sufficiently large charge densities for the stimulation and/or sufficiently low impedances for the recording therefore consists of operating with electrodes having a significant interface capacity. In the case of neurostimulation, this charge density may be all the more significant since the potential window of the electrode will be significant. Indeed, the larger the potential window, the larger a charge may be injected without dissociating the water present in the neighboring tissues.

The most frequently used electrode materials for the contemplated applications such as platinum and PtIr alloys, indium oxide, Tantalum/Ta₂O₅, titanium nitride and PEDOT have performances, reported in the article of S. F Cogan, entitled “Neural Stimulation and Recordings Electrodes”, Annu. Rev. Eng. 2008.10/275-309, and summarized in the table 1 below.

| | | | Potential window |
|---|---|---|---|
| | | Limiting injectable | (vs. AgAgCl |
| Materials | Mechanisms | charge (mC · cm⁻²) | reference) |
| Pt and Ptlr | Faradic/ | 0.05-0.15 | −0.6-0.8 |
| alloys | capacitive | | |
| Indium oxide | Faradic | 1-5 | −0.6-0.8 |
| Tantalum/Ta₂O₅ | Capacitive | ~0.5 | Not reported |
| Titanium nitride | Capacitive | ~1 | −0.9-0.9 |
| PEDOT | Capacitive | ~15 | −0.9-0.9 |

Unfortunately, all these materials generally have a significant stability problem and sometimes a biocompatibility problem.

The synthetic diamond which allows the solving of such a problem has also been considered as a potential electrode material for such applications.

Indeed, it is a chemically and biologically inert material which is the present subject of many scientific research studies in the medical field and in particular in that of implants and of neural query. It therefore seems to be very adapted for implantation in vivo in the long term. It also has excellent electrochemical properties, in particular has a large potential window of more than 3 V in aqueous media potentially allowing large injection of charge without dissociation of the surrounding medium, great resistance to corrosion, as well as a significant mechanical strength as compared with the other materials used. These electrochemical properties also seem to be adapted for electro-physiological applications ex-vivo. Generally, the growth of the diamond is accomplished on a substrate prepared beforehand for initiating growth by depositing by chemical vapor in a plasma containing hydrogen and a carbon source. The diamond films obtained after growth generally have a columnar poly-crystalline or nanocrystalline shape depending on the growth conditions. The dopant used generally boron which from concentrations typically greater than 10²¹ at.cm⁻³ gives it a quasi-metal conductivity with generally most performing electrochemical properties. This is referred to as a diamond doped with boron or [symbol] BDD [symbol] (Boron Doped Diamond) diamond. Thus MEA devices based on a diamond electrode BDD have been proposed for applications of medical implants or of microelectrode networks for electrophysiology and are described in the article of P. Bergonzo, et al., entitled <<3 d shaped mechanically flexible diamond microelectrode arrays for eye implant applications: The medinas project>> IRBM, 45(32):91-94, 2011, and the article of Michael W. Varney et al., entitled [symbol] Polycrystalline-diamond MEMS biosensors including neural microelectrode-arrays [symbol] , Biosensors, 1:118-113, 2011.

However, the poly-crystalline diamond doped with boron (BDD) stemming from columnar growth from diamond grains on a smooth substrate not having sufficient electrochemical properties for being efficiently used for neural stimulation or recording. In particular, the BDD diamond has an extremely small double layer electrochemical capacity of the order of 5 μF.cm⁻², which significantly limit the injectable charge density in spite of its large potential window. Further, the impedances of the BDD electrodes, traditionally measured at 1 kHz are relatively high, of the order of 1 MOhm in a saline medium buffered with phosphate ( [symbol] phosphate buffered saline [symbol] ) for electrodes with a diameter of 25 micrometers for example. The impedance of the electrode is partly dependent on the specific surface area of the electrode.

The technical problem is to find a biocompatible material for electrodes which increases their interfacial capacitance and reduces their electrochemical impedance when they are used for applications as mentioned earlier.

For this purpose, the object of the invention is a microelectrode for neural interfacing applications comprising a stack of a first substrate layer in a first biocompatible material, a second adhesion layer in a second material for initiating growth of synthetic diamond crystals, and a third layer in a third electrically conductive material, including poly-crystalline diamond doped with atoms comprised in the set formed by boron atoms, nitrogen atoms and phosphorus atoms,

the first, second, third layers having extension planes parallel to each other, characterized in that

the third material is a textured material which comprises a compact set, as a brush, of hollow or solid tubes, each consisting of at least one peripheral layer, radially external, of poly-crystalline diamond doped, radially deposited by high temperature growth, the tubes each having a first end attached to the second layer and a second free end, intended to be the active portion of the electrode,

the tubes being separated from each other by an empty space at their first ends and projecting their second ends in a direction away from the first and second layers substantially normal with respect to the extension plane of the second layer.

According to particular embodiments, the microelectrode includes one or several of the following features, taken alone or as a combination:
the tubes form a carpet in which, either all the tubes are substantially normal to the extension plane of the second layer and separated from each other in a regular way, or the tubes are grouped in bundles, separated from each other in a regular way and in which the second ends of the tubes of a same bundle are brought closer, or even touch each other;
each tube has a length comprised between 500 nm and 50 μm, and each tube has a section having a substantially constant diameter over the whole of its length or each tube has a variable section which decreases from its first end as far as its second end;
the second material is an adhesion material of the diamond suitable for being used as a diffusion barrier to a molten metal comprised in the set formed by nickel, cobalt, iron and nickel, iron, cobalt alloys, and the first material is a biocompatible material which may resist to growth conditions of the diamond, i.e. electrically insulating for example comprised in the set formed by SiO₂, Si₃N₄, quartz, glass, GaN, or electrically conductive for example comprised in the set formed by Pt, PtIr, Ti, TiN, TiPt alloys, diamond doped with boron;
the second material comprises the set formed by titanium nitride TiN, non-doped poly-crystalline diamond, poly-crystalline diamond doped with atoms comprised in the set formed by boron atoms, nitrogen atoms and phosphorus atoms, preferably poly-crystalline diamond doped with boron;
the method comprises a fourth layer in a fourth material consisting of doped polycrystalline diamond, the fourth layer covering the totality of the third layer based on tubes forming the second material;
the method comprises a fifth layer and a sixth layer,

the fifth layer being in a fifth metal material, forming an electric current socket of the third layer through the second layer when the latter is electrically conductive and/or of the fourth layer, the fifth layer being positioned on or above the first layer, either below the second layer when this layer is conductive, or in contact or at the periphery of the second and fourth layers independently of the electric conductivity of the second layer, the fifth layer having a contact area, forming an electric output terminal of the microelectrode and displaced from the third layer along the extension plane of the second layer,

the sixth layer being a biocompatible layer of passivation of the fifth layer covering the totality of the fifth layer except for its contact area forming the electric output terminal of the microelectrode.

The object of the invention is also a multi-electrode network for neural interfacing applications comprising a plurality of at least two microelectrodes defined above, developed and etched on a common stack of layers according to a distribution pattern on a planar surface.

The object of the invention is also a flexible implant for neural interfacing applications comprising:
a network of a multitude of microelectrodes as defined above, and
a matrix in a flexible polymeric material with a small thickness and a main two-dimensional extension including
a monolayer sheet, and

for each microelectrode, a single and different sheet with two layers covering the microelectrode while leaving exposed the seconds ends of its tubes and its contact area,

the whole of the sheets being gathered in a single part by the monolayer sheet, with or without through-hole.

The object of the invention is also a method for manufacturing a microelectrode for neural interfacing applications comprising the steps of

providing in a first step, a first dielectric substrate layer in a first biocompatible material, and then

in a second step, depositing a second adhesion layer in a second material for initiating growth of synthetic diamond crystals,

in a third step, making a third layer in a third electrically conductive material, including crystalline poly-crystalline diamond doped with atoms comprised in the set formed by boron atoms, nitrogen atoms and phosphorus atoms,

the first, second, third layers having planes with parallel extensions, characterized in that

the third material is a textured material which comprises a compact set, as a brush, of hollow or solid tubes, each consisting of at least one radially external peripheral layer, of doped poly-crystalline diamond, deposited radially by growth at a high temperature, the tubes each having a first end attached to the second layer and a second free end, intended to be the active portion of the electrode,

the tubes being separated from each other by an empty space at their first extensions and projecting their second ends in a direction away from the first and second layers substantially normal with respect to the extension plane of the second layer.

According to particular embodiments, the method for manufacturing a microelectrode includes one or several of the following features, taken alone or as a combination:
the manufacturing method comprises steps consisting of growing carbon nanotubes (CNT) on and from the second layer, and then in a fifth step, depositing one or several first layers of non-doped diamond nanoparticles on each of the carbon nanotubes, and then in a sixth step, depositing on the first non-doped diamond layer(s) by means of chemical vapor deposition assisted by plasma of the doped diamond by growing the doped diamond crystal until carbon nanotubes have been completely covered and their partial or complete etching with radical hydrogen contained in the plasma;
during the sixth step, a fourth layer in doped polycrystalline diamond is also formed so as to cover the totality of the third layer based on the tubes forming the second material;
the fourth step comprises:

a seventh step for depositing as a thin layer a metal comprised in the set formed by nickel, iron, cobalt, and their alloys notably FeNi, FeCoNi, preferably the nickel on the second layer, and then

an eighth de-wetting step by annealing the metal deposited as a thin layer in the seventh step in order to obtain regularly distributed metal drops over the second layer, and then

a ninth step in which carbon nanotubes are grown on and from metal drops being used as a catalyst;
the fifth step is applied:

either by a deposition said to be <<layer by layer>> consisting of successively depositing once or several times a layer of a poly-electrolyte polymer of positive or negative charge followed by a layer of diamond nanoparticles with an opposite charge, then immobilized on the carbon nanotubes by electrostatic attraction, the polymer being comprised in the set formed by Poly-(diallyldimethylammonium chloride) (PDDAC), polystyrene sulfonate (PSS); or

or by a deposition by [symbol] ink jet [symbol] type printing from a colloidal solution of non-doped nano-diamond; or

or by electrospray of diamond nanoparticles; and

the sixth step is applied by a chemical vapor deposition assisted by microwave plasma (PAVCD), or by radiofrequency chemical vapor deposition (RFCVD), or by a hot filament of a gas mixture comprising methane, dihydrogen and trimethyl of an atom comprised in the set formed by boron atoms, nitrogen atoms and phosphorus atoms;
the second material is comprised in the set formed by titanium nitride TiN, non-doped poly-crystalline diamond, poly-crystalline diamond doped with atoms comprised in the set formed by boron atoms, nitrogen atoms and phosphorus atoms, preferably poly-crystalline diamond doped with boron;
the second material is doped poly-crystalline diamond, and the second step is first applied by depositing with a spinner non-doped nano-diamond particles from an aqueous colloidal solution containing poly-vinyl alcohol, and then chemical vapor deposition assisted by plasma containing methane, dihydrogen and trimethyl of the doping atom;
the method comprises:

a tenth step for depositing a fifth layer, structured by a photo-lithographic method, the fifth layer being a fifth metal material, forming an electric current socket of the third layer through the second layer when the latter is electrically conductive and/or of the fourth layer, the fifth layer being positioned on the first layer, either above the second layer when the latter is conductive, or in contact or at the periphery of the second and fourth layers independently of the electric conductivity of the second layer, the fifth layer having a contact area, forming an electric output terminal of the microelectrode and displaced from the third layer along the extension plane of the first layer,

an eleventh step for depositing a sixth layer in a sixth biocompatible material and for passivation of the fifth layer, the sixth layer covering the totality of the fifth layer except for the contact area forming the electric output terminal of the microelectrode;
the first material is a biocompatible material which may resist to the growth conditions of the diamond, i.e. electrically insulating for example comprised in the set formed by SiO₂, Si₃N₄, quartz, glass, GaN, i.e. electrically conductive for example comprised in the set formed by Pt, PtIr, Ti, TiN, TiPt alloys, diamond doped with boron.

The invention will be better understood upon reading the description of several embodiments which will follow, only given as examples and made with reference to the drawings wherein:

FIG. 1 is a sectional view of a first embodiment of a microelectrode according to the invention;

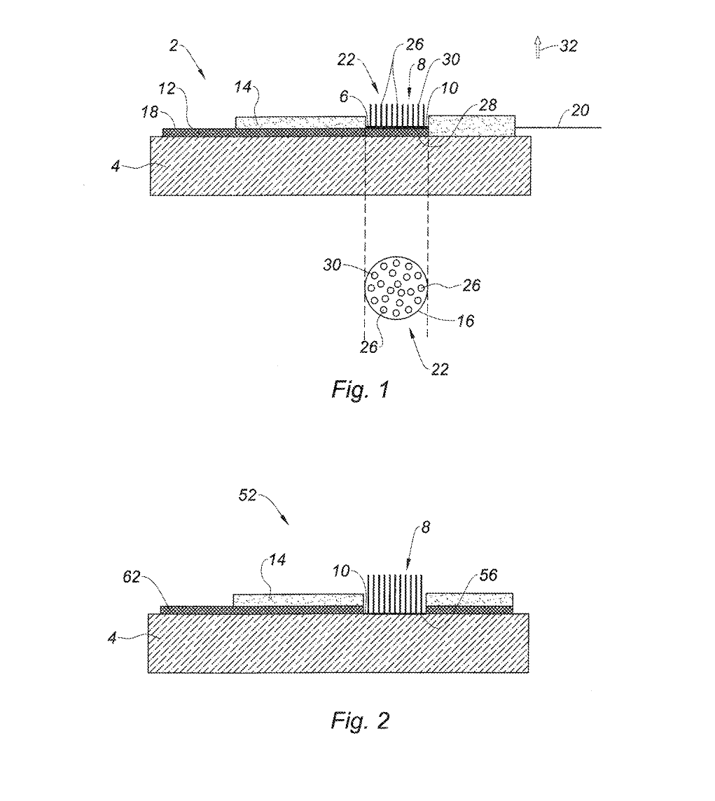

FIG. 2 is a sectional view of a second embodiment of a microelectrode according to the invention;

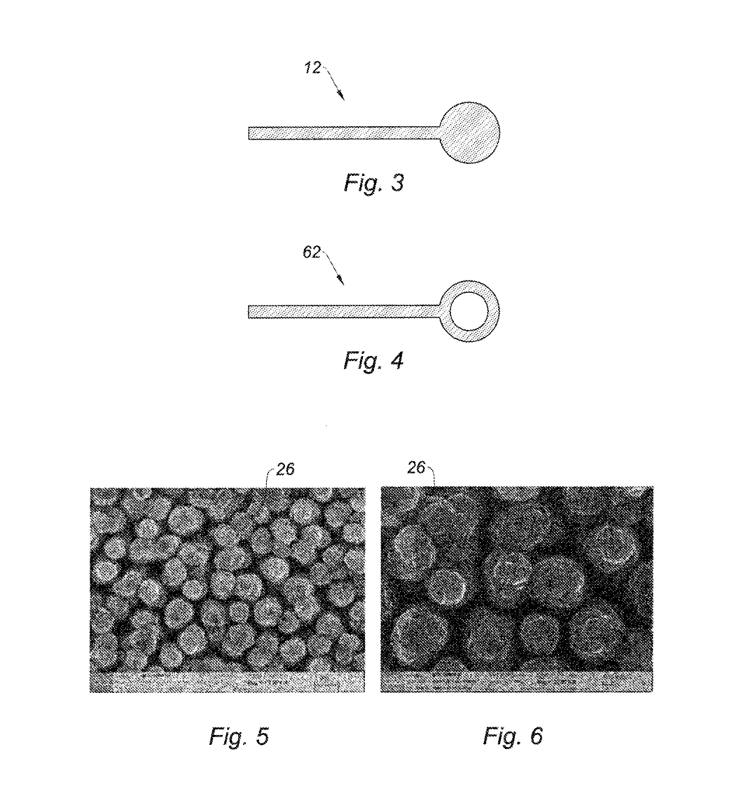

FIG. 3 is a view of the contacting of the microelectrode of FIG. 1;

FIG. 4 is a view of the contacting of the microelectrode of FIG. 2;

FIGS. 5 and 6 are top views in a scanning electron microscope of a same forest of tubes of the active portion of the microelectrodes of FIGS. 1 and 2 at different, respectively increasing magnifications,

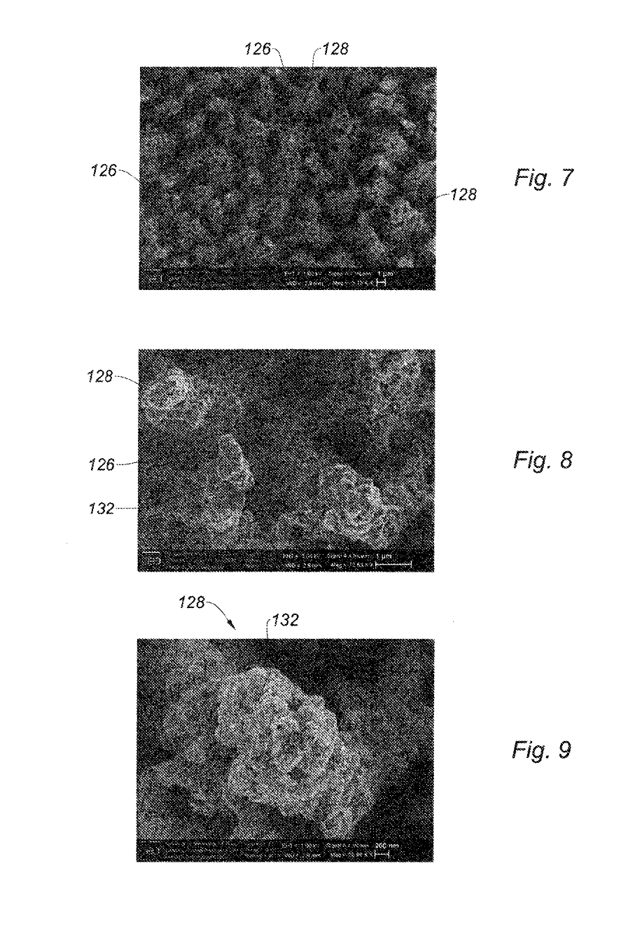

FIGS. 7, 8 and 9 are top views with a scanning electron microscope of a same forest of tubes of the active portion of a third embodiment of a microelectrode according to the invention at different, respectively increasing magnifications;

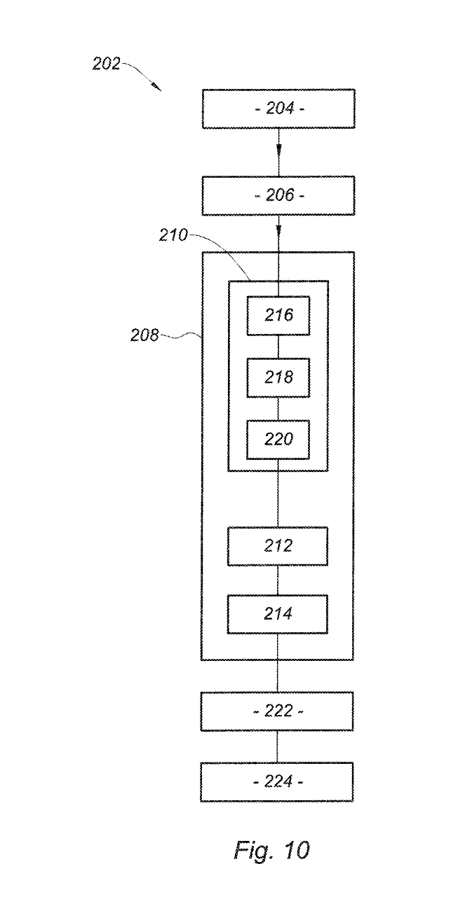

FIG. 10 is a general flow chart for manufacturing a microelectrode according to the invention, applicable to the manufacturing of the microelectrodes of FIGS. 1-2 and 5-9;

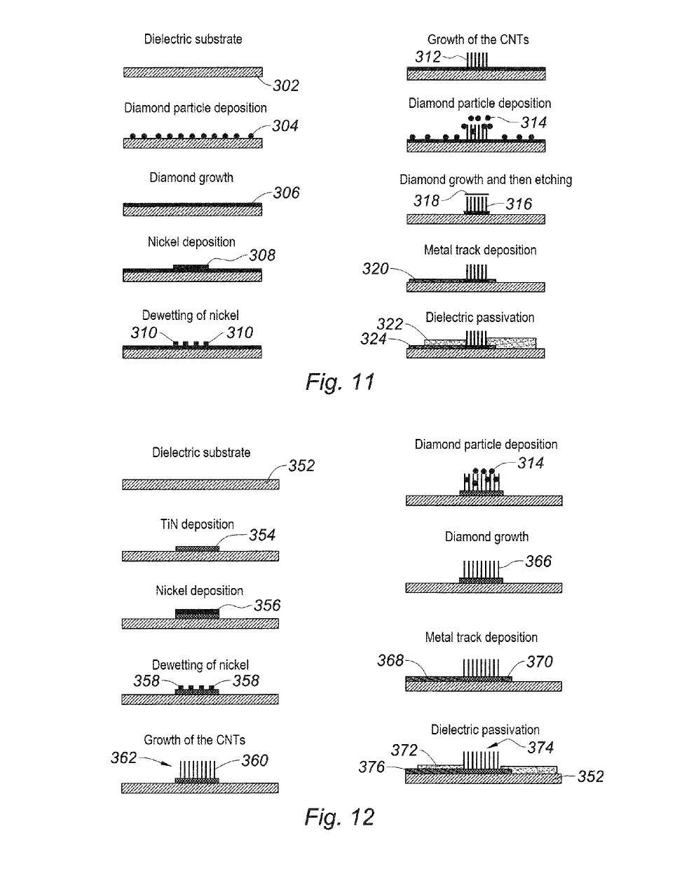

FIG. 11 is a view of a first embodiment of a multi-electrode network of the invention through different conditions of the network during its manufacturing;

FIG. 12 is a view of a second embodiment of a multi-electrode network of the invention through different conditions of the network during its manufacturing;

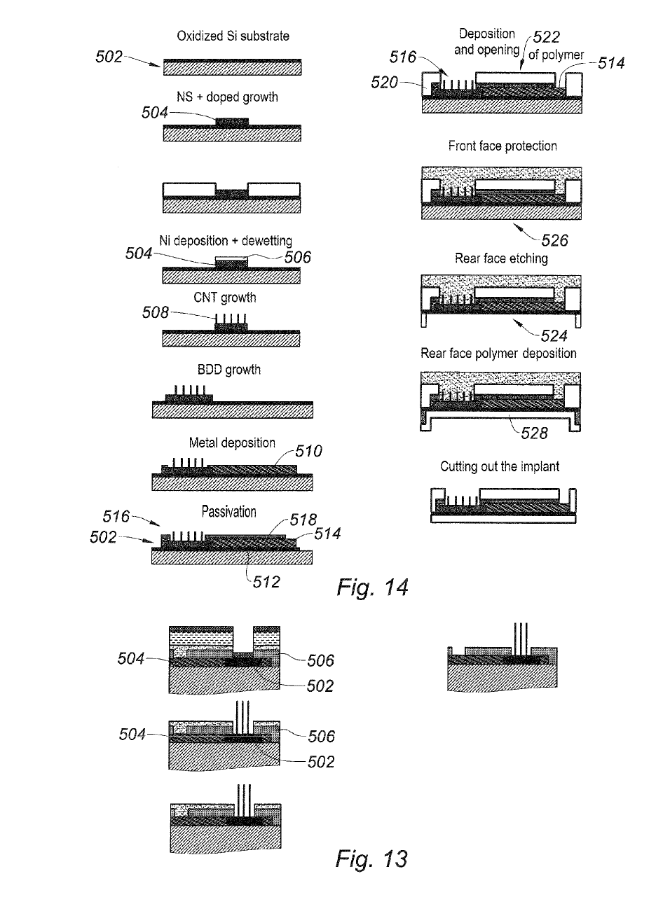

FIG. 13 is a view of a third embodiment of a multi-electrode network of the invention through different conditions of the network during its manufacturing;

FIG. 14 is a view of a fourth embodiment of a multi-electrode network of the invention through different conditions of the network during its manufacturing.

According to FIG. 1 and to a first embodiment, a microelectrode 2, configured for neural interfacing applications comprises a first substrate layer 4 in a first material, a second adhesion layer 6 in a second material for initiating growth of synthetic diamond crystals, a third layer 8 in a third material, a fourth layer 10 in a fourth material, a fifth layer 12 in a fifth material, and a sixth layer 14 in a sixth material.

The first, second, third layers 4, 6, 8 are mutually parallel extension planes.

The fifth layer 12 is positioned here between the first layer and the second adhesion layer of the third layer.

The fifth, second and third layers 12, 6, 8 share the same shape, here a circular shape, as that of an active portion 16 of the microelectrode 2 intended to come into contact with the biological tissue.

The fifth layer 12 forms a current socket of the third layer 8 through the second layer 6. The fourth layer 10 includes a contact area 18, forming an electric output terminal of the microelectrode 2 and displaced from the third 8 layer along the extension plane 20 of the second layer 6.

The sixth passivation layer 14 covers the totality of the fifth layer 12 except for its contact area 18 and of its portion covered by the second layer 6.

The first material is biocompatible and here electrically insulating. The first material is a material comprised in the set formed by SiO₂, Si₃N₄, quartz, glass, GaN, and generally any other biocompatible material which may resist to the growth conditions of the diamond.

The third material electrically conductive, and forming the active portion of the microelectrode, is synthetic poly-crystalline diamond, made electrically conductive by doping with atoms comprised in the set formed with boron atoms, nitrogen atoms and phosphorus atoms. Here, preferably, the second material is synthetic poly-crystalline diamond doped with boron (BDD).

The third material is also a textured material, i.e. having a structured surface, which comprises a compact set 22, as a brush, of hollow or solid tubes 26 with nanometric to micrometric dimensions.

The tubes 26 each consist at least on a peripheral external layer in poly-crystalline diamond, here doped with boron, and each have a first end 28, attached to the second layer 6, and a second free end 30.

The tubes 26 are separated from each other at their first ends 28 and project their second ends 30 in a direction 32 away from the first and second layers 4, 6, substantially vertical relatively to the extension plane 20 of the second layer 6.

Here, in a particular way, the tubes 26 form a carpet in which all the tubes are substantially vertical and separated from each other regularly. Each tube 26 substantially has the same length, and each tube includes a section having a substantially constant diameter over the whole of its length.

The diamond material is selected for the third material since it has excellent chemical inertia and high stability, as well as interesting electrochemical properties. Further, studies show that it is biologically inert and therefore is an excellent material for making implantable devices.

When the boron is used as a dopant at a concentration typically comprised between 10²¹ and 5.10²¹ at.cm⁻³, the electric conduction is also like a metal and the quality of the electrodes in terms of electrochemical performances is optimum.

The shape of the active portion of the microelectrode 2, i.e. of the carpet of tubes forming the third layer, here being cylindrical, the diameter of the third layer is comprised between 5 and 100 micrometers, and the height of the second layer is comprised between 500 nm and 50 μm.

The second material is an adhesion material of the tubes 26 of the third suitable material for being used as a diffusion barrier to molten metal comprised in the set formed by nickel, cobalt, iron and nickel, iron, cobalt alloys.

Here the second material is electrically conductive and forms a collector for the electric current provided by the third layer.

The second material when it is electrically conductive is titanium nitride TiN or poly-crystalline diamond made electrically conductive by doping with atoms comprised in the set formed by boron atoms, nitrogen atoms, and phosphorus atoms, and stemming from a standard columnar growth from diamond grains on a smooth substrate.

Here, preferably, the second material is synthetic poly-crystalline diamond doped with boron (BDD) and stemming from standard growth on a smooth substrate.

The fourth material consists of doped poly-crystalline diamond made to be conductive which covers the totality of the second layer based on the tubes forming the second material.

The fifth material generally consists of one or several metals and is isolated from the electrolytic solution formed at the interface of the biological tissue by the electrically insulating sixth layer 14.

The sixth material is a passivation, biocompatible and electrically insulating material.

According to FIG. 2, a second embodiment of the microelectrode 52 comprises the same elements as those described in the first embodiment of FIG. 1 except for the second layer 6 and the fifth layer 12, replaced with a second different layer and a fifth different layer, respectively designated by the references 56 and 62.

The second material of the second layer 56 is here non-doped poly-crystalline diamond and electrically insulating, stemming from a standard columnar growth from diamond grains on a smooth substrate.

The fifth metal layer 62, forms an electric current outlet of the third layer 8 through the fourth layer 10 while being positioned in contact and at the periphery of the second and fourth layers 56, 10.

Here, the presence of the fourth layer 10 is required because of the lack of electric conductivity of the second layer 56, the functions of which are limited to the adhesion of the tubes of the third material and to the diffusion barrier towards a molten metal, comprised in the set formed by nickel, cobalt, iron and nickel, iron, cobalt alloys and being used as a catalyst in a step for manufacturing the third material.

According to FIGS. 3 and 4, the shapes seen from above of the current outlets respectively formed with the fifth layer 12, 62 of the microelectrodes 2, 52 differ from each other in that the first fifth layer 12 in its portion for collecting the current has the shape of a solid disc, while the second fifth layer 52 has the shape of a ring fitting and covering the contour of the second and fourth layers.

The microelectrodes 2, 52 according to the invention, described in FIGS. 1 and 2, advantageously differ from the other standard electrodes known to one skilled in the art, for the following reasons:

(i) Just like standard electrodes BDD, the microelectrodes 2, 52 retain a larger potential window in aqueous media unlike the other electrodes known to one skilled in the art, which gives the possibility of obtaining large charge densities without degrading the solvent in the environment of the electrode.

(ii) In addition to the large potential window and this time unlike the standard BDD electrodes, the hybrid microelectrodes 2, 52 have an electric double layer capacity typically ten to five hundred times greater than that of standard BDD electrodes, which gives the possibility of both increasing the charge density as compared with a conventional diamond electrode and also considerably reducing the impedance of the electrode, which is particularly high in the case of a standard doped diamond.

(iii) Unlike standard electrode materials used in the targeted applications, the microelectrodes 2, 52 are extremely robust and stable.

(iv) The microelectrodes 2, 52 consisting in their active portion only of inert carbon are expected to have good acceptability of the tissues, in other words being biologically inert, as this was already demonstrated in the case of non-structured standard doped diamond.

According to FIGS. 5 and 6, the top views of the doped diamond tubes observed at different magnifications show a first arrangement of the tubes according to the first and second embodiments of FIGS. 1 and 2, i.e. embodiments in which the tubes 26 are substantially vertical and separate from each other in a regular way.

According to FIGS. 7, 8, and 9 and a third embodiment 102 of the microelectrode, derived from the first or second embodiments described in FIGS. 1 to 5, the tubes of the third layer, respectively designated by the numerical reference 126, are grouped in [symbol] bundles [symbol] 128, separated from each other in a regular way and wherein the second free ends 132 of the tubes 126 of a same bundle 128 are brought closer to each other, or even touch each other.

Each tube 126 has a variable section which decreases from its first end, attached to the third layer, as far as its second free end 132.

The applications targeted by the invention generally consist in the use of MEA networks of microelectrodes as described above. The networked electrodes on a same substrate will be electrically contacted individually.

In particular, for in-vivo applications, flexible implants may be made and configured for following the curves of the organs or the deformation of the latter. For example mention may be made of retinal implants which are introduced into the eye in order to stimulate cells of the retina.

Generally, a flexible implant for neural interfacing applications comprises an MEA network of at least two microelectrodes, developed and etched on a common stack of layers according to a distribution pattern on a planar surface, and a covering matrix.

The matrix, in a flexible polymer material of small thickness and with a two-dimensional main extension, includes a monolayer sheet, and for each microelectrode, a single and different sheet with two layers covering the microelectrode by leaving exposed the second ends of its tubes and its contact area. The bilayer sheets covering the microelectrodes are joined in one piece by the monolayer sheet, with or without any through-hole.

According to FIG. 10, a method 202 for manufacturing a microelectrode for neural interfacing applications as described above globally comprises a set of steps 204, 206, 208.

In a first step 204, a first substrate layer, in a first biocompatible material is provided.

The first material is a material, either electrically insulating comprised in the set formed by SiO₂, Si₃N₄, quartz, glass, GaN, or electrically conductive comprised in the set formed by Pt, PtIr, Ti, TiN, TiPt alloys, diamond doped with boron, and generally any other biocompatible conductive material which may support the temperatures for growth of synthetic diamond.

For example, here, the first material is assumed to be an electric insulator.

Next, in a following step 206, a second layer in a second material is deposited for initiating the growth of synthetic diamond crystals.

The second material is comprised in the set formed by titanium nitride TiN, non-doped poly-crystalline diamond, poly-crystalline diamond doped with atoms comprised in the set formed by boron atoms, nitrogen atoms and phosphorus atoms and stemming from standard growth on a smooth substrate.

Here, preferably, the second material is synthetic poly-crystalline diamond, doped with boron (BDD) and stemming from standard growth on a smooth substrate.

This non-structured standard diamond layer will have a double function. First it will act as a diffusion barrier for a metal catalyst used for growing carbon nanotubes (CNTs). Secondly, during the growth of the diamond on the CNTs, diamond from the non-structured third layer and diamond growing on the CNTs will [symbol] merge [symbol] , thereby promoting better adhesion of the second structured layer on the substrate.

Next, in a third step 208, a third layer in a third material, electrically conductive, is made. The third material consist in synthetic poly-crystalline diamond, made to be electrically conductive by doping with atoms comprised in the set formed by boron atoms, nitrogen atoms, and phosphorus atoms.

When the boron is used for example as a dopant at a concentration typically comprised between 10²¹ and 5.10²¹ at.cm⁻³, the electric conduction is then quasi-metal conduction and the quality of the electrodes in terms of electrochemical performances is optimum.

Here, preferably, the third material is synthetic poly-crystalline diamond doped with boron (BDD).

The third material is a textured material which comprises a compact set, as a brush, of hollow or solid tubes of nanometric to micrometric dimensions. The tubes consist in doped poly-crystalline diamond, here doped with boron, and each have a first end, attached to the second layer, and a second free end, intended to form the active portion of the microelectrode.

The tubes are separated from each other at their first ends and project their second ends into a direction away from the first and second layers, substantially vertical with respect to the extension plane of the first layer.

The first, second, third layers are deposited so that their extension planes are mutually parallel.

The third material is made preferably by means of an original method consisting of growing diamond on a set of sacrificial carbon nanotubes on at least one portion of their individual structure.

The third step 208 comprises successive execution of a fourth step 210, of a fifth step 212 and of a sixth step 214.

The fourth step 210 consist of growing Carbon NanoTubes (CNTs), which are sacrificial on at least one portion of their individual structure, on and from the second layer. Next, in the fifth step 212, one or several first layers of non-doped synthetic diamond nanoparticles are deposited on each of the carbon nanotubes (CNTs). Next, in the sixth step 214, by a chemical vapor deposition method assisted by plasma of the doped diamond, made to be conductive by doping, is deposited on the first layer(s) of non-doped diamond by growing the doped diamond crystal until it completely covers the carbon nanotubes and partial or complete etching of the latter with radical hydrogen contained in the plasma.

The fourth step 210 comprises a seventh step 216, an eighth step 218 and a ninth step 220, successively carried out.

In the seventh step 216, a thin layer of a metal catalyst, comprised in the set formed by nickel, iron, cobalt, and their alloys, notably FeNi, FeCoNi, and preferably nickel (Ni), is deposited on the second layer in order to obtain on the substrate droplets with a nanometric size. Such a deposition is well mastered by one skilled in the art. The metal catalyst is used for catalyzing the growth of CNTs. Since the metal catalyst tends to diffuse into the substrate during the annealing or during the growth of CNTs, the second layer, deposited during the second step 206, acts as a diffusion barrier. As described above, a layer of synthetic diamond is preferred since it moreover promotes adhesion of the third layer of structured diamond.

The eighth step 218 consist of de-wetting by annealing the metal catalyst deposited on a thin layer in the seventh step 216 for obtaining metal droplets of a nanometric size regularly distributed on the second layer according to a pattern corresponding to the shape of the active portion of the microelectrode.

Once the metal catalyst layer is deposited and de-wetted on the substrate, during the ninth step 220, the growth of the carbon nanotubes (CNTs) on and from metal drops used as a catalyst is achieved by a chemical vapor deposition method (CVD). The length of the CNTs may typically vary from 500 nm to 5 micrometers, and will preferably be comprised between 1 and 2 micrometers. These CNTs will be of single sheet or multi-sheet types. The CNTs may be misoriented (in <<spaghetti>> form) or preferentially vertically aligned on the substrate.

Once the carbon nanotubes CNTs have been deposited on the substrate, the fifth step 212 during which non-doped diamond nanoparticles are in turn deposited on the CNTs, this is achieved for example according to one of the three following methods.

In a first method, a [symbol] layer by layer [symbol] deposition is achieved consisting of successively depositing a layer of a poly-electrolyte polymer of a positive or negative charge followed by a layer of diamond nanoparticles with opposite charge, then immobilized on the CNTs by electrostatic attraction. The polymers currently used for this task are Poly-(diallyldimethylammonium chloride) (PDDAC) or further Polystyrene sulfonate (PSS). This stack of layers may be repeated several times in order to increase the particle density on the CNTs.

In a second method, a deposition from a printing system of the <<ink jet>> type is achieved from a colloidal solution of non-doped nanodiamond.

In a third step, electrospraying of diamond nanoparticles is carried out.

Depending on the method and on the deposition conditions, the CNTs may retain their initial geometrical aspect or be agglomerated so as to form bundles.

The sixth step 214 is applied by chemical vapor deposition assisted by a microwave plasma (PAVCD), or by a radiowave plasma (RFCVD), or with a hot filament of a gas mixture comprising methane, dihydrogen and trimethyl boron, when boron is used, under adequate conditions known to one skilled in the art. The growth of doped diamond, preferentially with boron at a concentration typically comprised between 10²¹ and 5.10²¹ at.cm⁻³ will be continued until complete covering and partial or complete disappearance of the CNTs which will for a large part be etched by radical hydrogen present in the plasma.

In all the applications of the sixth step 214, at least one or several of the most external sheets of the CNTs are sacrificed.

It should be noted that there exist several methods for synthesizing diamond, from among which the chemical vapor deposition methods assisted by microwave plasma (PACVD being the acronym of Plasma Assisted Chemical Vapor Deposition) or RF (Radio Frequency) or with a hot filament are the most used by experts in the field of diamond synthesis. The PACVD method for example generally consist of growing diamond grains of nanometric size (2-100 nm), on a substrate placed in a growth PACVD reactor typically operating at 80-4,000 Watts in a gas mixture comprising at least a mixture of methane and dihydrogen with an adequate proportion. During the growth, the temperature of the substrate is commonly comprised between 400 and 900° C. Some diamond powder may be deposited on the substrate before the growth step, but there also exist other possible surface treatments which may initiate diamond growth. During the growth, the diamond grains will grow on the substrate in the CVD plasma until a continuous poly-crystalline diamond is obtained. In order to dope the diamond, a source of atoms comprised from among a source of boron atoms, a source of nitrogen atoms, a source of phosphorus atoms, is generally introduced into the plasma during the growth, for example in the case of boron as diborane or trimethyl boron gas. The dissociated boron in the plasma will then be incorporated into the diamond crystal or be substituted for a carbon atom in the crystal.

An alternative of the invention will consist of depositing this structured diamond layer on suitable supports for making electrodes which may be used in certain implantable medical devices. Indeed, many electrode systems implantable to this day (e.g. cochlear implants) are not manufactured in clean rooms with micro-fabrication techniques but rather by mechanical mounting of electrodes of diverse shapes generally larger with typical dimensions of the order of a few millimeters. These electrodes may for example be platinum discs, platinum-iridium, etc. Thus, the structured diamond material, i.e. the third layer, may be deposited on such supports for increasing the mechanical, electrochemical, and biocompatibility performances before mounting in the implantable devices.

The structured material obtained after growing the diamond provides two important functions for the microelectrode: first of all it allows a considerable increase in the value of the electric capacitance of the electrode, typically by a ratio varying from 10 to 500. Moreover, it increases the specific surface area of the electrode since, unlike non-structured polycrystalline diamond, it has a roughness which contributes to reducing the total impedance of the electrode. Unlike other materials known to one skilled in the art, this material also has a large potential window, greater than or equal to 2.5 V in an aqueous medium, i.e. comparable with that of non-structured polycrystalline diamond. It is also very stable chemically.

As already seen in FIGS. 1 to 2 and 5 to 9, the rough diamond appearing as a forest of diamond pillars forms the active portion of the microelectrode. An electric current outlet being used as a connection between the active portion of the microelectrode and an external terminal is required for being able to use the electrode properly.

As already seen in FIGS. 1 and 2, the current outlet is placed either between the second layer and the substrate, or at the periphery and in contact with the third layer and a fourth layer.

The fourth layer is mandatory when the material of the second layer is an electric insulator. It consists in doped polycrystalline diamond, made to be conductive and covers the totality of the third layer based on the tubes forming the third material.

The material of the electric outlet generally consist of one or several metals or metal alloy, most often gold or platinum deposited on a metal being used as an adhesion layer on the substrate, e.g. chromium or titanium.

Further, the electric outlet is isolated from the electrolytic solution forming the interface with a biological tissue, by a passivation layer consisting of a dielectric material such as for example silicon dioxide SiO₂, silicon nitride Si₃N₄, polymers such as SUB, Polyimide, Parylene.

In order to produce the current outlet and the passivation layer, the method 202 described in FIG. 10 further comprises a tenth step 222 for depositing a fifth layer and a third step 224 for depositing a passivation layer on the electric outlet. In the tenth step 222, a fifth layer, structured by a photo-lithographic method in a un fifth metal material, is deposit so as to form an electric current socket of the third layer through the second layer when the letter is electrically conductive and/or the fourth layer. The fifth layer is positioned on the first layer, either underneath the second layer when the latter is conductive, or independently of the electric conductivity of the second layer in contact and at the periphery of the second and fourth layers. The fifth layer includes a contact area, forming an output electric terminal of the microelectrode and shifted away from the third layer along the extension plane of the first layer.

In the eleventh step 224, a sixth biocompatible passivation layer of the fifth layer is deposited so as to cover the totality of the fifth layer except for the contact area forming the electric output terminal and of the active surface of the electrode.

Thus, a first advantage of the invention is that these hybrid electrodes retain a large potential window in aqueous media comparable with that of the ordinary BDD diamond electrodes, unlike the other electrodes known to one skilled in the art. This gives the possibility of obtaining large charge densities of the order of 0.1 mC.cm⁻² to 5 mC.cm⁻² (at 100 mV.s⁻¹) without degrading the solvent in the environment of the electrode.

Further, this time unlike the standard BDD electrodes, the electrode has a double layer capacitance typically greater than 200 μF.cm⁻², i.e. forty times greater than that of standard BDD electrodes, which in particular gives the possibility of increasing the charge density relatively to a diamond electrode. This high capacitance coupled with greater roughness and therefore a larger specific surface area of the electrode also contributes to considerably reducing the impedance of the electrode, particularly high in the case of non-structured diamond (poly-crystalline diamond as extracted from the growth reactor), by a factor 15 to 100 depending on the texturation.

According to FIG. 11, a first embodiment of a method for manufacturing an MEA multi-electrode network is described through different views each corresponding to a different phase of the condition of the network in the manufacturing process.

Here, a glass substrate 302 of the molten silica type, i.e. <<fused silica>>, is used for manufacturing the MEA network. The electrodes are deposited on this electrically insulating substrate. Any other insulating substrate (eg. Si/SiO₂ oru Si/Si₃N₄, quartz, etc.) having good stability at the growth temperatures of synthetic diamond and of CNTs could have been used. By [symbol] good stability [symbol] is meant a substrate which does not soften and/or does not deform under the effect of heat.

On this substrate 302, a deposit of diamond particles 304 ( [symbol] nanopowder [symbol] ) of a nanometric dimension is deposited. The diamond nanopowder used will preferentially be so-called [symbol] detonation [symbol] powder, because of its small size (5 to 15 nanometers for the primary nano powder). Alternatively, the nanopowder may also be obtained by milling diamond powder of a coarser size. The average diameter of interest of the diamond nano powder will typically be of the order of 1 to 100 nm. In both cases, the core of the nano powder in a large majority consists of sp3-hybridized carbon. The diamond nano powder may be used crude, or after purification in the case of the detonation powder. Here, we use nano powder of an average de diameter of 20 nm obtained by milling. Several methods for depositing these particles are known to one skilled in the art, like the one consisting of depositing them with the spinner from an aqueous colloidal solution of particles containing an adequate proportion of polyvinyl alcohol, which is accomplished in this example.

Once the diamond particles are deposited on the substrate, growth of the diamond is initiated in a CVD growth reactor, in a plasma containing methane, hydrogen and trimethyl boron in adequate proportions. The pressure in the growth chamber is comprised between 20 and 40 mbars. The plasma is maintained from a microwave energy source with a power comprised between 2 and 4 kW. Under the conditions above, the temperature of the substrate is comprised between 600 and 800° C. A poly-crystalline film of boron-doped diamond 306 is then obtained with a thickness comprised between typically 100 nm and 1,000 nm.

On this first layer of poly-crystalline diamond, a structured deposit of nickel 308 is achieved by photolithography by means of the so-called <<lift-off>> method known to one skilled in the art. These nickel structures define locations where the structured diamond electrodes will be found later on.

This nickel layer is then [symbol] de-wetted [symbol] by a heat treatment known to one skilled in the art in order to obtain nanometric structures of nickel 310 (droplets) which will be used for catalyzing the growth of a forest of carbon nanotubes (CNTs).

The substrate is then placed in a reactor for growth of carbon nanotubes. Nanotubes 312 vertically oriented are then manufactured by a method known to one skilled in the art. Here the carbon nanotubes have a length of about 2 micrometers.

Once the growth of the carbon nanotubes is accomplished, a nanodiamond layer 314 is deposited either on the totality of the substrate, by the [symbol] layer by layer [symbol] method described earlier, by using PDDAC as an adhesion polymer.

Once this deposit has been made, new growth of diamond doped with boron is achieved under the same conditions as earlier until a diamond layer of typically 500 nm on carbon nanotubes is obtained.

Next, the diamond structures 316 then appearing in the definitive form of a forest of diamond pillars are protected by a metal mask or by a photosensitive resin 318 once again by the photo lithographic method known to one skilled in the art. Next, the non-protected diamond layer is etched by a method of the ionic etching type RIE (Reactive Ion Etching) until complete disappearance of the non-protected diamond layer. Next, the protective mask is removed by etching.

It should be noted that during the step for growing CNTs, there exist conditions which gives the possibility of etching the diamond not covered with Ni (etching with an NH₃ plasma which is basically used in certain methods for growing CNTs). The diamond not covered with nickel is etched, the latter covered with nickel will be used as a base for growing CNTs. The latter protecting the diamond from the etching during their growth. This avoids the lithography-RIE etching steps mentioned just before).

A metal contact outlet 320 is then carried on the studs. Here, the contacts are made from a stack of Ti/Pt layers. The Ti is used here for promoting adhesion of Pt on the substrate. These deposits will be structured by photo lithographic methods known to one skilled in the art. Here, a metal ring will thus be deposited at the periphery of the diamond electrodes in order to leave the centre of the electrode in diamond exposed.

A so called passivation layer 322 is finally deposited on the substrate by leaving a non-covered area 324 on the microelectrode and on the contacts. This is the aperture 324 which defines the active area of the electrode. According to FIG. 12, a second embodiment of a method for manufacturing an MEA multi-electrode network is described through different views each corresponding to a different phase of the condition of the network in the manufacturing process.

Here a glass substrate 352 of the “fused silica” type is once again used for manufacturing the MEA network. The microelectrodes will be deposited on this electrically insulating substrate. Like in the previous example, any other insulating substrate (e.g. Si/SiO₂ or Si/Si₃N₄, quartz, etc.) having good stability at the temperatures for growing the synthetic diamond might have been used.

On this substrate, a TiN layer 354 is locally deposited at the future location of the microelectrodes. On these TiN structures, is deposited a nickel layer 356 which is then de-wetted by a heat treatment in order to obtain nickel nano drops 358.

And then growth of CNTs 360 is achieved from nickel nano drops 356 on the TiN layer 354 until a forest 362 of CNTs with lengths comprised between 1 and 2 micrometers is obtained.

Next, from a colloidal solution of diamond nano particles 364, these diamond nanoparticles 364 are locally deposited on the forests 362 of CNTs by means of a printing technique of the ink jet type.

Once the diamond particles 364 deposited on the CNTs, growth of the diamond is initiated in a CVD growth reactor, in a plasma containing methane, hydrogen and trimethyl boron in adequate proportions. The pressure in the growth chamber is comprised between 20 and 40 mbars. The plasma is maintained from a microwave energy source with a power comprised between 2 and 4 kW. Under the conditions above, the temperature of the substrate is comprised between 600 and 800° C. A polycrystalline film of structured boron-doped diamond 366 is then obtained in the place of the CNTs like in the previous example.

A metal contact outlet 368 is then carried out on the studs 364 formed by the etched layer, called a second layer in FIGS. 1 and 2. Here the contact 368 is made from a stack of Ti/Pt layers. The Ti is used here for promoting the adhesion of Pt on the substrate. These deposits will be structured by photo lithographic methods known to one skilled in the art. Here a metal ring will thus be deposited at the periphery of the diamond electrodes in order to leave the center of the electrode in diamond exposed.

A so-called passivation layer 372 is finally deposited on the substrate 352 and the metal contacts 368 by leaving an area 374 not covered over the electrode and on the contact outlet 372. It is this aperture 374 which defines the active area of the electrode.

It should be noted that when it is possible to deposit TiN on Pt or PtIr discs for preparing a suitable degree of roughness for the surface of the discs, a method for producing a multi-electrode network as described in FIG. 13 may be contemplated.

According to FIG. 13, a third embodiment of a method for manufacturing a network of multiples electrodes is described through different views each corresponding to a different phase of the condition of the network in the manufacturing process.

First of all, a second adhesion layer 502 in BDD, an outlet 504 of metal contacts which supports the high temperatures and the passivation 506 are made. The whole is encapsulated in a sacrificial metal layer 508 which supports the temperature, does not catalyze the carbon nanotubes (CNTs) and does not promote growth of CNTs. Next an opening lithography gives the possibility of etching the stack formed by the sacrificial metal layer, the passivation layer as far as the second BDD layer. A <<lift-off>> of nickel is achieved, followed by the growth of carbon nanotubes CNTs and then by a deposition of a deposit of BDD diamond on the carbon nanotubes CNTs. The sacrificial metal is selectively removed, the aperture of the passivation layer having been provided during the deposition of the passivation layer clearing the contact areas shifted from the contact outlet.

According to FIG. 14, a fourth embodiment of a method for manufacturing a network of multiple electrodes forming a flexible implant is described through different views each corresponding to a different phase of the condition of the network in the manufacturing process.

From a silicon substrate oxidized beforehand 502, bases 504 of a diamond electrode are made. A deposit of nickel 506 is achieved on these bases 504 of electrodes and then a step for de-wetting nickel is carried out. The growth of the nanotubes 508 (CNTs) is achieved and these nanotubes 508 are covered with diamond nanoparticles. Growth of diamond doped with boron (BDD) is carried out in order to cover all the nanotubes and to sacrifice them. Next a layer 510 of metal Cr/Au is deposited for defining the tracks 512, the output terminals 514 and for achieving contact on the diamond electrodes 516, a single track and a single terminal being illustrated in FIG. 15. A nitride layer 518 for passivation is deposited on the substrate 502 and the contact connection 510, and locally open so as to define the microelectrodes 516 and the output terminals 514. Next a polymer 520 is deposited on the front face 522 and locally opened in order to access the microelectrodes 516 and the contact or output terminals 514. The front face 522 of the wafer is protected and an aperture 524 on the rear face 526 is made in the silicon substrate as far as the oxide layer 524. And then a second deposit 528 of polymer is carried out on the open rear face 526. All that remains is to cut out the shape of the implant, for example by means of a laser.

1. A microelectrode for neural interfacing applications
comprising a stack of a first layer of substrate in a first biocompatible material, a second adhesion layer in a second material for initiating the growth of synthetic diamond crystals, and a third layer in a third electrically conductive material, including poly-crystalline diamond doped with atoms comprised in the set formed by boron atoms, nitrogen atoms and phosphorus atoms,
the first, second, third layers (1, 6, 8) having mutually parallel extension planes, characterized in that
the third material is a textured material which comprises a compact set, as a brush, of hollow or solid tubes, each consisting of at least one peripheral layer, radially external, of doped poly-crystalline diamond, radially deposited by growth at high temperature, the tubes each having a first end attached to the second layer and a second free end, intended to be the active portion of the electrode,
the tubes being separated from each other by an empty space at their first ends and projecting their second ends in a direction away from the first and second layers substantially normal with respect to the extension plane of the second layer.

2. The microelectrode according to claim 1, characterized in that the tubes form a carpet in which, either all the tubes are substantially normal with respect to the extension plane of the second layer and separated from each other regularly, or the tubes are grouped in bundles, separated from each other regularly and wherein the second ends of the tubes of a same bundle are brought closer to each other, or even touch each other.

3. The microelectrode according to claim 1, wherein each tube has a length comprised between 500 nm and 50 μm, and
each tube has a section having a substantially constant diameter over the whole length or each tube has a variable section which decreases from its first end as far as its second end.

4. The microelectrode according to claim 1, wherein the second material is an adhesion material for diamond suitable for being used as a diffusion barrier to a molten metal comprised in the set formed by nickel, cobalt, iron and nickel, iron, cobalt alloys, and
the first material is a biocompatible material which may resist to the growth conditions of the diamond, either electrically insulating comprised for example in the set formed by SiO₂, Si₃N₄, quartz, glass, GaN, or electrically conductive for example comprised in the set formed by Pt, PtIr, Ti, TiN, TiPt alloys, diamond doped with boron.

5. The microelectrode according to claim 4, wherein the second material is comprised in the set formed by titanium nitride TiN, non-doped poly-crystalline diamond, poly-crystalline diamond doped with atoms comprised in the set formed by boron atoms, nitrogen atoms and phosphorus atoms, preferably poly-crystalline diamond doped with boron.

6. The microelectrode according to claim 1, comprising a fourth layer in a fourth material consisting of doped polycrystalline diamond, the fourth layer covering the totality of the third layer at the base of the tubes forming the second material.

7. The microelectrode according to claim 1, comprising a fifth layer and a sixth layer,
the fifth layer being in a fifth metal material, forming an electric current outlet of the third layer through the second layer when the latter is electrically conductive and/or of the fourth layer, the fifth layer being positioned on or above the first layer, either underneath the second layer when the latter is conductive, or in contact and at the periphery of the second and fourth layers independently of the electric conductivity of the second layer, the fifth layer having a contact area, forming an electric output terminal of the microelectrode and shifted from the third layer along the extension plane of the second layer,
the sixth layer being a biocompatible layer for passivation of the fifth layer covering la totality of the fifth layer except for its contact area forming the electric output terminal of the microelectrode.

8. A network of a multitude of microelectrodes for neural interfacing applications comprising a plurality of at least two microelectrodes defined according to claim 1, developed and etched on a stack of layers common according to a distribution pattern on a planar surface.

9. A flexible implant for neural interfacing applications comprising:
a network of a multitude of microelectrodes defined according to claim 8 wherein, and
a matrix in a flexible polymeric material of small thickness with a two-dimensional main extension including
a monolayer sheet, and
for each microelectrode, a single and different sheet with two layers covering the microelectrode while leaving exposed the second ends of its tubes and of its contact area,
the whole of the sheets being join together in a single part by the monolayer sheet, with or without any through-hole.

10. A method for manufacturing a microelectrode for neural interfacing applications comprising the steps consisting of
providing in a first step a first dielectric substrate layer in a first biocompatible material, and then
in a second step depositing a second adhesion layer in a second material for initiating growth of synthetic diamond crystals,
in a third step manufacturing a third layer in a third electrically conductive material, including crystalline poly-crystalline diamond doped with atoms comprised in the set formed by boron atoms, nitrogen atoms and phosphorus atoms,
the first, second, third layers having parallel extensions planes, characterized in that
the third material is a textured material which comprises a compact set as a brush, of hollow or solid tubes, each consisting of at least one peripheral layer, radially external, of doped poly-crystalline diamond, deposited radially by growth at high temperature, the tubes each having a first end attached to the second layer and a second free end, intended to be the active portion of the electrode,
the tubes being separated from each other by an empty space at their first ends and projecting their second ends in a direction away from the first and second layers substantially normal with respect to the extension plane of the second layer.

11. The manufacturing method according to claim 10, wherein the third step comprises the steps consisting of
in a fourth step, growing carbon nanotubes (CNT) on and from the second layer, and then
in a fifth step, depositing one or several first layers of non-doped diamond nanoparticles on each of the carbon nanotubes, and then
in a sixth step, depositing on the first non-doped diamond layer(s) by chemical vapor deposition assisted by plasma of the doped diamond by glowing the doped diamond crystal until complete cover of the carbon nanotubes and partial or complete etching with radical hydrogen contained in the plasma.

12. The manufacturing method according to claim 10, wherein during the sixth step a fourth layer in doped polycrystalline diamond is also formed so as to cover the totality of the third layer at the base of the tubes forming the second material.

13. The manufacturing method according to claim 11, wherein
the fourth step comprises:
a seventh step for depositing as a thin layer a metal comprised in the set formed by nickel, iron, cobalt, and their alloys notably FeNi, FeCoNi, preferably nickel on the second layer, and then
an eighth step for de-wetting by annealing of the metal deposited as a thin layer in the seventh step in order to obtain metal drops regularly distributed over the second layer, and then
a ninth step in which carbon nanotubes are grown on and from metal drops being used as a catalyst.

14. The manufacturing method according to claim 11, wherein the fifth step is applied:
either with a so-called <<layer by layer>> deposition consisting of
successively depositing once or several times a layer of a poly-electrolyte polymer of positive or negative charge followed by a layer of diamond nanoparticles of opposite charge, then immobilized on carbon nanotubes by electrostatic attraction, the polymer being comprised in the set formed by poly-(diallyldimethylammonium chloride) (PDDAC), polystyrene sulfonate (PSS); or
by depositing by <<ink jet>> type printing from a colloidal solution of non-doped nanodiamond; or
by electrospray of diamond nanoparticles; and
the sixth step is applied by Plasma-Assisted Chemical Vapor Deposition (PAVCD) or by Radio Frequency Chemical Vapor Deposition or by a hot filament in a gas mixture comprising methane, dihydrogen and a trimethylated atom comprised in the set formed by boron atoms, nitrogen atoms and phosphorus atoms.

15. The manufacturing method according to claim 11, wherein
the second material is comprised in the set formed by titanium nitride TiN, non-doped polycrystalline diamond, polycrystalline diamond doped with atoms comprised in the set formed by boron atoms, nitrogen atoms and phosphorus atoms, preferably polycrystalline diamond doped with boron.

16. The manufacturing method according to claim 15, wherein
the second material is doped polycrystalline diamond, and
the second step is first applied by depositing with a spinner non-doped nano-diamond particles from an aqueous colloidal solution containing polyvinyl alcohol and then a plasma-assisted chemical vapor deposition containing methane, dihydrogen and trimethylated dopant atom.

17. The manufacturing method according to claim 11, comprising
a tenth step for depositing a fifth layer structured by a photo-lithographic method, the fifth layer being in a fifth metal material, forming an electric current outlet of the third layer through the second layer when the latter is electrically conductive and/or of the fourth layer, the fifth layer being positioned on the first layer, either below the second layer when the latter is conductive, or in contact or at the periphery of the second and fourth layers independently of the electric conductivity of the second layer, the fifth layer having a contact area forming an electric output terminal of the micro-electrode and offset from the third layer along the extension plane of the first layer,
an eleventh step for depositing a sixth layer in a sixth biocompatible material and for passivation of the fifth layer, the sixth layer covering the totality of the fifth layer except for its contact area forming the electric output terminal of the micro-electrode.

18. The manufacturing method according to claim 11, wherein
the first material is a biocompatible material which may withstand the growth conditions of the diamond, i.e. electrically insulating, for example comprised in the set formed by SiO2, Si3N4, quartz, glass, GaN, i.e. electrically conductive, for example comprised in the set formed by Pt, PtIr, Ti, TiN, TiPt alloys, diamond doped with boron.
